(19) United States
(12) Patent Application Publication
(10) Pub. No.: US 2007/0087025 A1
(43) Pub. Date: Apr. 19, 2007

(54) Reiteratively layered medical devices and method of preparing same

(76) Inventors: Anthony Fitzhugh, Frederick, MD (US); Peiwen Cheng, Santa Rosa, CA (US)

Correspondence Address: KIRKPATRICK & LOCKHART PRESTON GATES ELLIS LLP, 1900 MAIN STREET, SUITE 600, IRVINE, CA, 92614-7319, US

(21) Appl. No.: 11/056,323

(22) Filed: Feb. 10, 2005

Related U.S. Application Data
(63) Continuation of application No. 10/490,991, now abandoned.
(60) Provisional application No. 60/325,049, filed on Sep. 26, 2001.

(51) Int. Cl. A61K 31/727; A61F 2/00; B05D 3/02

(52) U.S. Cl. 424/423; 427/2.26

(57) ABSTRACT

A method for preparing a nitric oxide-releasing substrate that includes contacting an amine-functionalized silane with a substrate, contacting at least one additional amine-functionalized silane with the substrate, and contacting the substrate with nitric oxide, and repeating these steps if and as desired to produce a coating of the desired thickness as well as quantity and duration of nitric oxide-release.

### FIELD OF THE INVENTION

This invention pertains to a nitric oxide-releasing amine-functionalized polysilane coated medical devices, a method for making the same and methods of using same.

### BACKGROUND OF THE INVENTION

Nitric oxide (NO) is a simple diatomic molecule that plays a diverse and complex role in cellular physiology. It is known that NO is a powerful signaling compound and cytotoxic/cytostatic agent found in nearly every tissue of the human body, including endothelial cells, neural cells, and macrophages. NO has been implicated recently in a variety of bioregulatory processes, including normal physiological control of blood pressure, angiogenesis, and thrombosis, as well as neurotransmission, cancer, and infectious diseases. See, e.g., Moncada, “Nitric Oxide,” J. Hypertens. Suppl. 12(10): S35-39 (1994); Moncada et al., “Nitric Oxide from L-Arginine: A Bioregulatory System,” Excerpta Medica, International Congress Series 897 (Elsevier Science Publishers B.V.: Amsterdam, 1990); Marletta et al., “Unraveling the Biological Significance of Nitric Oxide,” Biofactors 2: 219-225 (1990); Ignarro, “Nitric Oxide. A Novel Signal Transduction Mechanism for Transcellular Communication,” Hypertension 16: 477-483 (1990); Hariawala et al., “Angiogenesis and the Heart: Therapeutic Implications,” J.R. Soc. Med. 90(6): 307-311 (1997); Granger et al., “Molecular and Cellular Basis of Myocardial Angiogenesis,” Cell. Mol. Biol. Res. 40(2): 81-85 (1994); Chiueh, “Neuroprotective Properties of Nitric Oxide,” Ann. N.Y. Acad. Sci. 890: 301-311 (1999); Wink et al., “The Role of Nitric Oxide Chemistry in Cancer Treatment,” Biochemistry (Moscow) 63(7): 802-807 (1998); Fang, F. C., “Perspectives Series: Host/Pathogen Interactions. Mechanisms of Nitric Oxide-Antimicrobial Activity,” J. Clin. Invest. 99(12): 2818-25 (1997); and Fang, F. C., “Nitric Oxide and Infection,” (Kluwer Academic/Plenum Publishers: New York, 1999).

Glyceryl trinitrate and sodium nitroprusside are two examples of vasodilators that currently enjoy widespread clinical use and whose pharmacological actions result from their metabolic conversion in situ to NO-releasing species. See, e.g., Ignarro et al., J. Pharmocol. Exp. Ther. 218: 739-749 (1981); Ignarro, Annu. Rev. Pharmacol. Toxicol. 30: 535-560 (1990); and Kruszyna et al., Chem. Res. Toxicol. 3: 71-76 (1990). In addition, other agents have been described in the literature which release NO spontaneously or following metabolic conversion of their parent or prodrug forms. See, e.g., Drago, ACS Adv. Chem. Ser. 36: 143-149 (1962); Longhi and Drago, Inorg. Chem. 2: 85 (1963); Schönafinger, “Heterocyclic NO prodrugs,” Farmaco 54(5): 316-320 (1999); Hou et al., “Current trends in the Development of Nitric Oxide Donors,” Curr. Pharm. Des. 5(6): 417-441 (1999); Muscara et al., “Nitric Oxide. V. Therapeutic Potential of Nitric Oxide Donors and Inhibitors,” Am. J. Physiol. 276(6, Pt. 1): G1313-1316 (1999); Maragos et al., “Complexes of NO with Nucleophiles as Agents for the Controlled Biological Release of Nitric Oxide. Vasorelaxant Effects,” J. Med. Chem. 34: 3242-3247 (1991); Fitzhugh et al., “Diazeniumdiolates: pro- and antioxidant applications of the ‘NONOates,’” Free Radic. Biol. Med. 28(10): 1463-1469 (2000); Saavedra et al., “Diazeniumdiolates (Formerly NONOates) in Cardiovascular Research and Potential Clinical Applications,” Nitric Oxide and the Cardiovascular System (Humana Press: Totowa, N.J., 2000); and Yamamoto et al., “Nitric oxide donors,” Proc. Soc. Exp. Biol. Med. 225(3): 200-206 (2000).

NO-donor compounds can exert powerful tumoricidal and cytostatic effects. Such effects are attributable to NO's ability to inhibit mitochondrial respiration and DNA synthesis in certain cell lines. In addition to these bioregulatory properties, NO may arrest cell migration. These effects are apparently not limited to NO-donor compounds as macrophages can also sustain high levels of endogenous NO production via enzymatic mechanisms. Similar inhibitory effects have also been observed in other cells. See, e.g., Hibbs et al., “Nitric Oxide: A Cytotoxic Activated Macrophage Effector Molecule,” Biochem. and Biophys. Res. Comm. 157: 87-94 (1988); Stuehr et al., “Nitric Oxide. A Macrophage Product Responsible for Cytostasis and Respiratory Inhibition in Tumor Target Cells,” J. Exp. Med. 169: 1543-1555 (1989); Zingarelli, et al., “Oxidation, Tyrosine Nitration and Cytostasis Induction in the Absence of Inducible Nitric Oxide Synthase,” Int. J. Mol. Med. 1(5): 787-795 (1998); Yamashita et al., “Nitric Oxide is an Effector Molecule in Inhibition of Tumor Cell Growth by rIFN-gamma-activated Rat Neutrophils,” Int. J. Cancer 71(2): 223-230 (1997); Garg et al., “nitric oxide-Generating Vasodilators Inhibit Mitogenesis and Proliferation of BALB/C 3T3 Fibroblasts by a Cyclic GMP-Independent Mechanisms,” Biochem. and Biophys. Res. Comm. 171: 474-479 (1990); and Sarkar et al., “Nitric Oxide Reversibly Inhibits the Migration of Cultured Vascular Smooth Muscle Cells,” Circ. Res. 78(2): 225-30 (1996).

Medical research is rapidly discovering a number of potential therapeutic applications for NO-releasing compounds/materials, particularly in the fields of vascular surgery and interventional cardiology. For example, fatty deposits may build up on the wall of an artery as plaque. Over time as additional material is added, the plaque thickens, dramatically narrowing the cross-sectional area of the vessel lumen in a process known as arteriosclerosis. Blood flow to the heart muscle is compromised resulting in symptoms ranging from intermittent chest pain to easy fatigability. In an effort to reduce such symptoms and improve blood flow, patients with this condition may opt to undergo a procedure known as coronary artery bypass grafting (CABG). In a typical CABG procedure, a portion of a vein is removed from the leg. Sections of the vein are then used to bypass the site(s) of plaque-induced coronary artery narrowing. CABG involves a major surgical procedure wherein the patient's chest is opened to facilitate the operation, as a result, it carries with it appreciable morbidity and mortality risks. However, bypassing the site(s) of greatest narrowing with a grafted vein substantially alleviates the chest pain and fatigue that are common in this condition while reducing the risk of acute arterial blockage. A less invasive and increasingly common procedure for treating plaque-narrowed coronary arteries is called percutaneous transluminal coronary angioplasty (PTCA) (also known as balloon angioplasty). In PTCA, a catheter is inserted into the femoral artery of the patient's leg and threaded through the circulatory system until the site of coronary vessel occlusion is reached. Once at the site, a balloon on the tip of the catheter is inflated which compresses the plaque against the wall of the vessel. The balloon is then deflated and the catheter removed. PTCA results in dramatic improvement in coronary blood flow as the cross-sectional area of the vessel lumen is increased substantially by this procedure. However, common complications of this procedure include thrombus formation at the site of PTCA-treatment, vessel rupture from overextension, or complete collapse of the vessel immediately following deflation of the balloon. These complications can lead to significant alterations in blood flow with resultant damage to the heart muscle.

To limit many of the problems associated with PTCA-treatment, cardiologists will frequently insert a small tubular device known as a stent. The stent serves as a permanent scaffold for maintaining vessel patency following deflation and removal of the balloon-tipped catheter from the artery. Since the stent is a permanent implant, its insertion can cause the vessel wall at the site of PTCA-injury to respond in a complex multi-factorial process known as restenosis. This process is initiated when thrombocytes (platelets) migrate to the injury site and release mitogens into the injured endothelium. Clot formation or thrombogenesis occurs as activated thrombocytes and fibrin begin to aggregate and adhere to the compressed plaque on the vessel wall. Mitogen secretion also causes the layers of vascular smooth muscle cells below the site of injury (neointima) to over proliferate, resulting in an appreciable thickening of the injured vessel wall. Within six months of PTCA-treatment roughly 30 to 50% of patients will exhibit significant or complete re-occlusion of the vessel.

Nitric oxide has recently been shown to dramatically reduce thrombocyte and fibrin aggregation/adhesion and smooth muscle cell hyperplasia while promoting endothelial cell growth (Cha et al., “Effects of Endothelial Cells and Mononuclear Leukocytes on Platelet Aggregation,” Haematologia (Budap) 30(2): 97-106 (2000); Lowson et al., “The Effect of Nitric Oxide on Platelets When Delivered to the Cardiopulmonary Bypass Circuit,” Anest. Analg. 89(6): 1360-1365 (1999); Riddel et al., “Nitric Oxide and Platelet Aggregation,” Vitam. Horm. 57: 25-48 (1999); Gries et al., “Inhaled Nitric Oxide Inhibits Human Platelet Aggregation, P-selectin expression, and Fibrinogen Binding In Vitro and In Vivo,” Circulation 97(15): 1481-1487 (1998); and Lüscher, “Thrombocyte-vascular Wall Interaction and Coronary Heart Disease,” Schweiz ‘Med. Wochenschr’ 121(51-52): 1913-1922 (1991)). NO is one of several “drugs” under development by researchers as a potential treatment for the restenotic effects associated with intracoronary stent deployment. However, because the cascade of events leading to irreparable vessel damage can occur within seconds to minutes of stent deployment, it is essential that any anti-restenotic “drug” therapy be available at the instant of stent implantation. Also, it is widely thought that such therapy may need to continue for some time afterwards as the risk of thrombogenesis and restenosis persists until an endothelial lining has been restored at the site of injury.

In theory, one approach for treating such complications involves prophylactically supplying the PTCA-injury site with therapeutic levels of NO. This can be accomplished by stimulating the endogenous production of NO or using exogenous NO sources. Methods to regulate endogenous NO release have primarily focused on activation of enzymatic pathways with excess NO metabolic precursors like L-arginine and/or increasing the local expression of nitric oxide synthase (NOS) using gene therapy. U.S. Pat. Nos. 5,945,452, 5,891,459, and 5,428,070 describe the sustained NO elevation using orally administrated L-arginine and/or L-lysine while U.S. Pat. Nos. 5,268,465, 5,468,630, and 5,658,565 describe various gene therapy approaches. Other various gene therapy approaches have been described in the literature. See, e.g., Smith et al., “Gene Therapy for Restenosis,” Curr. Cardiol. Rep. 2(1): 13-23 (2000); Alexander et al., “Gene Transfer of Endothelial Nitric Oxide Synthase but not Cu/Zn Superoxide Dismutase restores Nitric Oxide Availability in the SHRSP,” Cardiovasc. Res. 47(3): 609-617 (2000); Channon et al., “Nitric Oxide Synthase in Atherosclerosis and Vascular Injury: Insights from Experimental Gene Therapy,” Arterioscler. Thromb. Vasc. Biol. 20(8): 1873-1881 (2000); Tanner et al., “Nitric Oxide Modulates Expression of Cell Cycle Regulatory Proteins: A Cytostatic Strategy for Inhibition of Human Vascular Smooth Muscle Cell Proliferation,” Circulation 101(16): 1982-1989 (2000); Kibbe et al., “Nitric Oxide Synthase Gene Therapy in Vascular Pathology,” Semin. Perinatol. 24(1): 51-54 (2000); Kibbe et al., “Inducible Nitric Oxide Synthase and Vascular Injury,” Cardiovasc. Res. 43(3): 650-657 (1999); Kibbe et al., “Nitric Oxide Synthase Gene Transfer to the Vessel Wall,” Curr. Opin. Nephrol. Hypertens. 8(1): 75-81 (1999); Vassalli et al., “Gene Therapy for Arterial Thrombosis,” Cardiovasc. Res. 35(3): 459-469 (1997); and Yla-Herttuala, “Vascular Gene Transfer,” Curr. Opin. Lipidol. 8(2): 72-76 (1997). However, these methods have not proved clinically effective in preventing restenosis. Similarly, regulating endogenously expressed NO using gene therapy techniques such as NOS vectors remains highly experimental. Also, there remain significant technical hurdles and safety concerns that must be overcome before site-specific NOS gene delivery will become a viable treatment modality.

The exogenous administration of gaseous nitric oxide is not feasible due to the highly toxic, short-lived, and relatively insoluble nature of NO in physiological buffers. As a result, the clinical use of gaseous NO is largely restricted to the treatment of neonates with conditions such as persistent pulmonary hypertension (Weinberger et al., “The Toxicology of Inhaled Nitric Oxide,” Toxicol. Sci. 59(1), 5-16 (2001); Kinsella et al., “Inhaled Nitric Oxide: Current and Future Uses in Neonates,” Semin. Perinatol. 24(6), 387-395 (2000); and Markewitz et al., “Inhaled Nitric Oxide in Adults with the Acute Respiratory Distress Syndrome,” Respir. Med. 94(11), 1023-1028 (2000)). Alternatively, however, the systemic delivery of exogenous NO with such prodrugs as nitroglycerin has long enjoyed widespread use in the medical management of angina pectoris or the “chest pain” associated with atherosclerotically narrowed coronary arteries. There are problems with the use of agents such as nitroglycerin. Because nitroglycerin requires a variety of enzymes and cofactors in order to release NO, repeated use of this agent over short intervals produces a diminishing therapeutic benefit. This phenomenon is called drug tolerance and results from the near or complete depletion of the enzymes/cofactors needed in the blood to efficiently convert nitroglycerin to a NO-releasing species. By contrast, if too much nitroglycerin is initially given to the patient, it can have devastating side effects including severe hypotension and free radical cell damage.

Because of problems associated with the systemic delivery of NO, there has been a recent shift towards identifying agents/materials capable of directly releasing NO or other antirestenotic agents over a prolonged period directly at the site of PTCA-vascular injury. As a result, there exists a substantial need for a stent comprised of or coated with a material capable of continuously releasing NO from the instant of contact with a blood field to days or weeks following its deployment in a coronary artery. Such a device potentially represents an ideal means of treating the restenosis that frequently accompanies the implantation of a stent into a coronary artery. See, e.g., U.S. Pat. Nos. 6,087,479 and 5,650,447, U.S. Patent Application No. 2001/0000039, and PCT No. WO 00/02501, that detail prior art approaches to developing NO-releasing coatings for metallic stents and other medical devices.

Diazeniumdiolates comprise a diverse class of NO-releasing compounds/materials that are known to exhibit sufficient stability to be useful as therapeutics. Although discovered more than 100 years ago by Traube et al., Liebigs Ann. Chem. 300:81-128 (1898), the chemistry and properties of diazeniumdiolates have been extensively reinvestigated by Keefer and co-workers, as described in U.S. Pat. Nos. 4,954,526, 5,039,705, 5,155,137, 5,212,204, 5,250,550, 5,366,997, 5,405,919, 5,525,357, and 5,650,447, and in J. A. Hrabie et al., J. Org. Chem. 58: 1472-1476 (1993), and incorporated herein by reference.

Because many NO-releasing diazeniumdiolates have been prepared from amines, one potential approach for treating PTCA-associated restenosis is to coat the device with a suitably diazeniumdiolated amine-functionalized polymeric material. U.S. Pat. No. 5,405,919, for example, describes several biologically acceptable, amine-functionalized polyolefin-derived polymers. However, there are a number of problems associated with polyolefin-based coatings. They are prone to fractures as the coating is stressed during procedures such as stent expansion. Were such fractures to occur, it might cause particulate fragments from the coating to be released into the lumen of the overstretched vessel, ultimately lodging downstream in much narrower arteriolae and capillaries and compromising blood flow to those portions of the heart muscle that are supplied by the affected artery. Additionally, polyolefin-based and -coated medical devices tend to be more prone to the development of biofilms and device-related infections. These problems suggest that polyolefin-based materials may not be appropriate for uses in which permanent in situ implantation is desired. By contrast, metallic medical devices have repeatedly been shown to exhibit bio- and hemocompatibility properties that are superior to many polyolefin-based materials. See, Palmaz, “Review of Polymeric Graft Materials for Endovascular Applications,” J. Vasc. Interv. Radiol. 9(1 Pt. 1): 7-13 (1998); Tepe et al., “Covered Stents for Prevention of Restenosis. Experimental and Clinical Results with Different Stent Designs,” Invest. Radiol. 31(4): 223-229 (1996); Fareed, “Current Trends in Antithrombotic Drug and Device Development,” Semin. Thromb. Hemost. 22(Suppl. 1): 3-8 (1996); Bolz et al., “Coating of Cardiovascular Stents with a Semiconductor to Improve Their Hemocompatibility,” Tex. Heart Inst. J. 23(2): 162-166 (1996); De Scheerder et al., “Biocompatibility of Polymer-Coated Oversized Metallic Stents Implanted in Normal Porcine Coronary Arteries,” Atherosclerosis 114(1): 105-114 (1995); and Libby et al., “Ultrasmooth Plastic to Prevent Stent Clogging,” Gastrointest. Endosc. 40(3): 386-387 (1994). More recently, quite dramatic improvements in bio- and hemocompatibility have also been observed in medical devices coated with certain polymeric materials (e.g., silicone, hydrogel, heparin-, albumin-, phosphorylcholine-functionalized polymers and the like). See, e.g., Malik et al., “Phosphorylcholine-Coated Stents in Porcine Coronary Arteries. In Vivo Assessment of Biocompatibility,” J. Invasive Cardiol. 13(3): 193-201 (2001); Tsang et al., “Silicone-Covered Metal Stents: An In Vitro Evaluation for Biofilm Formation and Patency,” Dig. Dis. Sci. 44(9): 1780-1785 (1999); Kuiper et al., “Phosphorylcholine-coated Metallic Stents in Rabbit Illiac and Porcine Coronary Arteries,” Scand. Cardiovasc. J. 32(5): 261-268 (1998); and McNair, “Using Hydrogel Polymers for Drug Delivery,” Med. Device Technol. 7(10): 16-22 (1996).

Beyond the type of material used to coat the medical device, methods for precisely dosing NO have not yet been perfected with any of the NO-releasing diazeniumdiolated compounds/materials that have been developed to date. When exposed to hydrogen ion (i.e., proton) donors such as, for example, water or physiological fluids, most diazeniumdiolates bearing unshielded and unprotected [(NO)NO]⁻ groups rapidly break down to produce a “burst” of NO. This initial surge or burst of NO is typically followed by a steady but diminishing rate of release until the entire NO content of the material has been exhausted. For most diazeniumdiolated compounds, such processes are complete within minutes to a few hours of the initial NO burst.

Accordingly, there remains a need for an NO-releasing medical device suitable for use in the treatment of various medical indications and which are compatible with the animal body, including the human body and internal organs, blood vessels, tissues and cells. Desirably such devices are capable of the sustained release of NO for periods lasting days to a few weeks or longer. The invention described herein provides for the preparation of such coated medical devices. These and other advantages of the present invention, as well as additional inventive features, will be apparent from the description of the invention provided below.

### BRIEF SUMMARY OF THE INVENTION

The invention provides a method of preparing a polysilane-coated nitric oxide-releasing substrate and the polysilane-coated nitric oxide-releasing substrate. By “substrate” it is meant to include any material capable of reacting with silanes. Exemplary substrate materials include metal, glass, ceramic, plastic, rubber, natural fibrous materials, synthetic fibrous materials, or any combination thereof. Preferably, the substrate is a metal, glass, ceramic, plastic or rubber substrate. More preferably, the substrate is metal.

By “nitric oxide-releasing” is meant that nitric oxide is released from the substrate under physiological conditions. Physiological conditions include, for example, physiological buffers, blood, bodily fluids, tissues and the like.

Generally, the method of the invention includes the deposition and bonding of amine-functionalized polysilanes onto the surface of a substrate and contacting the substrate with NO. Amine-functionalized polysilanes can be deposited as a single layer or as multiple layers. The repeated, or reiterative, deposition of the polysilanes used can be made to form a multi-layer and coated substrate. When reacted with NO, the single or multiply layered substrate in accordance with the invention yields a coated substrate capable of releasing NO under physiological conditions. Advantageously, the substrate constitutes, or is part of, a medical device.

Specifically, the preferred method includes hydrolyzing an amine-functionalized silane in the presence of a hydrolyzing reagent. The hydrolyzing reagent can be any reagent capable of hydrolyzing the silane. Preferably, the hydrolyzing reagent is an aqueous solvent. It is believed that an aqueous solvent hydrolyzes the silane to form mono- and oligomeric silane. Advantageously, the aqueous solvent is water. The hydrolyzed amine-functionalized is reacted with a substrate to form a single layer substrate. This single layer coated substrate can be reacted with NO to form a nitric oxide-releasing coated substrate. Preferably, the single layer substrate is reacted with at least one additional hydrolyzed amine-functionalized silane to form a multi-layer substrate to enhance the nitric oxide capacity of the coated substrate. It will also be appreciated that the additional hydrolyzed amine-functionalized silane can be the same as the amine-functionalized that is hydrolyzed, or it can be different. The choice of silanes adds to the viability of the invention. The multi-layer substrate is reacted with nitric oxide gas to form a reiteratively layered nitric oxide-releasing substrate.

Optionally, nitric oxide releasing functional groups can be reacted with the amine-functionalized silane. The method is reiterative in that the deposition and bonding of the amine-functionalized silane to the substrate can be repeated as many times as deemed necessary in order to produce the desired coating thickness. In this regard, the invention is tunable in that the thickness of the substrate coating directly correlates with the quantity of NO that can be bonded to or absorbed by the substrate, e.g., stored, and ultimately released from the surface of the modified substrate under physiological condictions. In some cases, it is desirable to release lower levels of NO and a thin amine-functionalized polysilane coating is applied to the substrate; and in other cases, a more prolonged release of NO is desired and a thick coating is applied to the substrate.

The invention further provides polysilane-coated nitric oxide-releasing substrates, such as medical devices. Such devices are preferably prepared by the methods described herein. The term “medical device” refers to any device, product, equipment or material having surfaces that contact tissue, blood, or other bodily fluids in the course of their use or operation, which fluids are found in or are subsequently used in patients or animals. Medical devices include, for example, extracorporeal devices for use in surgery, such as blood oxygenators, blood pumps, blood storage bags, blood collection tubes, blood filters including filtration media, tubing used to carry blood and the like which contact blood which is then returned to the patient or animal. Medical devices also include endoprostheses implanted in a human or animal body, such as stents, pacemaker, pacemaker leads, heart valves, pulse generator, cardiac defibrillator, cardioverter defibrillator, spinal stimulator, brain and nerve stimulator, introducer, chemical sensor, and the like, that are implanted in blood vessels or the heart. Medical devices also include devices for temporary intravascular use such as catheters, guide wires, amniocentesis and biopsy needles, cannulae, drainage tubes, shunts, sensors, transducers, probes and the like which are placed into the blood vessels, the heart, organs or tissues for purposes of monitoring, repair or treatment. Medical devices also include prostheses such as hips or knees as well as artificial hearts. Medical devices also include implants, specula, irrigators, nozzles, calipers, forceps, retractors, vascular grafts, personal hygiene items, absorbable and nonabsorbable sutures, wound dressings, and the like.

### DETAILED DESCRIPTION OF THE INVENTION

The invention provides substrates, such as medical devices, that are capable of releasing nitric oxide when in use, but that are otherwise inert to nitric oxide release. In particular, nitric oxide is bound to a substrate coated with a multi-layered amine-functionalized silane; more particularly, the amine-functionalized silane is derived from a polysiloxane. Alternatively, nucleophile residues or substances may be bound to the coated substrate, followed by diazeniumdiolation with nitric oxide. The nucleophile residue may be separate from the substrate, part of the substrate, or present as pendant groups attached to molecules and/or polymers covalently linked to the substrate. The term “bound” as used herein includes covalent bonds, ionic bonds, van der Waal forces, hydrogen bonding, electrostatic bonding, and all other methods for attaching nitric oxide to a substrate.

The term “diazeniumdiolation,” as used herein, refers to the process of contacting a nucleophile residue with NO gas to produce a nitric oxide-releasing nucleophile residue complex containing the [N(O)NO] subunit. Reaction of the amine-functionalized polysilane with NO can occur by any method known in the art. Diazeniumdiolation can occur either through the neat exposure to NO gas or by immersing the coated substrate in an organic solvent and then exposing the solution to NO. Typical organic solvents include, for example, acetonitrile, diethyl ether, tetrahydrofuran, dioxane or mixtures thereof. In the solvent system, the NO gas can be bubbled into the solvent containing the coated substrate or added under mild or elevated pressure using typical equipment and methods known in the art. Additionally, any temperature can be used so long as it allows for the formation of at least one nitric oxide-releasing diazeniumdiolate group.

One preferred embodiment of the invention provides a method for preparing a nitric oxide-releasing substrate. Specifically, the method includes: (a) hydrolyzing an amine-functionalized silane in an aqueous reagent; (b) contacting the hydrolyzed amine-functionalized silane with the substrate to form a single layer substrate; (c) contacting the single layer substrate with at least one additional hydrolyzed amine-functionalized silane to form a multi-layer substrate; and (d) contacting the multi-layer substrate with nitric oxide gas.

The substrate can be any material capable of reacting with silanes. The substrate can be of any form, including a sheet, a fiber, a tube, a fabric, an amorphous solid, an aggregate, dust, or the like. Exemplary substrate materials include metal, glass, ceramic, plastic, rubber, natural fibrous materials, synthetic fibrous materials, or any combination thereof. Natural materials include cotton, silk, linen, hemp, wool, and the like. More preferably, the substrate is a metal, glass, ceramic, plastic or rubber substrate. Most preferably, the substrate is metal. Advantageously, the substrate comprises a biocompatible material.

Exemplary metal substrates include stainless steel, nickel, titanium, iron, tantalum, aluminum, copper, gold, silver, platinum, zinc, silicon, magnesium, tin, alloys, coatings containing any of the above and combinations of any of the above. Also included are such metal substrates as galvanized steel, hot dipped galvanized steel, electrogalvanized steel, annealed hot dipped galvanized steel and the like. Preferably, the metal substrate is stainless steel.

Exemplary glass substrates include soda lime glass, strontium glass, borosilicate glass, barium glass, glass-ceramics containing lanthanum, and combinations thereof.

Exemplary ceramic substrates include boron nitrides, silicon nitrides, aluminas, silicas, and combinations thereof.

Exemplary plastic substrates and synthetic fibrous materials include acrylics, acrylonitrile-butadiene-styrene, acetals, polyphenylene oxides, polyimides, polystyrene, polypropylene, polyethylene, polytetrafluoroethylene, polyvinylidene, polyethylenimine, polyesters, polyethers, polylactones, polyurethanes, polycarbonates, polyethylene terephthalate, as well as copolymers thereof and combinations thereof.

Exemplary rubber substrates include silicones, fluorosilicones, nitrile rubbers, silicone rubbers, fluorosilicone rubbers, polyisoprenes, sulfur-cured rubbers, isoprene-acrylonitrile rubbers, and combinations thereof. Silicones, fluorosilicones, polyurethanes, polycarbonates, polylactones, and mixtures or copolymers thereof are preferred plastic or rubber substrates because of their proven bio- and hemocompatibility when in direct contact with tissue, blood, blood components, or bodily fluids.

Exemplary natural fibrous materials include cotton, linen, silk, hemp, wool, and combinations thereof.

Other exemplary substrates include those described in WO 00/63462, and incorporated herein by reference, as well as combinations of the above-mentioned substrates.

The amine-functionalized silanes encompassed within the scope of the invention include any suitable silane compound capable of being bound to the substrate and that may be further derivatized with NO or nitric oxide-releasing functional groups to confer NO-releasing capabilities. Exemplary amine-functionalized silane compounds include those disclosed and described in, for example, U.S. Pat. Nos. 6,024,918, 6,040,058, 6,001,422, and 6,072,018, and PCT Nos. WO 99/37721 and WO 00/63462, and are incorporated herein by reference. Preferably, the amine-functionalized silane is any suitable compound, such as hydrolyzable silane compounds, having a reactive amino or polyaminoalkyl moiety attached to a di- or trialkoxysiloxane nucleus, including bis-aminosilanes having di- and trisubstituted silyl groups, wherein the hydrolyzable substituents include functionalities such as alkoxy, aryloxy, acyloxy, amine, chlorine and the like.

The aminosilanes and bis-aminosilanes can be described generally by the formulae shown below:

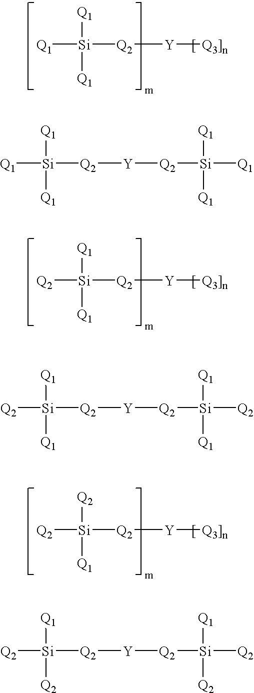

wherein m is either 1 or 2, n=(2−m), and each Q₁ is the same or different and is an organofunctional moiety. Exemplary organofunctional moieties include alkoxy, aryloxy, acyloxy, amine, halo or derivatives thereof. The organofunctional moiety Q₁ can be unsubstituted or substituted C₁₋₂₄ aliphatic, unsubstituted or substituted C₃₋₁₂ olefinic, unsubstituted or substituted C₃₋₂₄ heterocycloalkyl, unsubstituted or substituted C₃₋₂₄ cycloalkyl, unsubstituted or substituted C₃₋₃₀ aryl, unsubstituted or substituted benzyl, unsubstituted or substituted phenyl, unsubstituted or substituted benzylcarbonyl, unsubstituted or substituted phenylcarbonyl, or saccharides. The moiety Y is an amine-containing moiety. Exemplary amine-containing moieties include, for example, 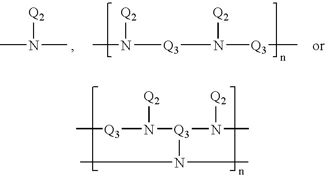
wherein n is an integer of 2-100. Each of the moieties Q₂ and Q₃ can be the same or different and are organic or inorganic moieties. Exemplary organic or inorganic moieties Q₂ and Q₃ include nitric oxide-releasing functional groups as described herein, hydrogen, unsubstituted or substituted C₁₋₂₄ aliphatic, unsubstituted or substituted C₃₋₁₂ olefinic, unsubstituted or substituted C₃₋₂₄ cycloalkyl, unsubstituted or substituted C₃₋₂₄ heterocycloalkyl, unsubstituted or substituted C₃₋₃₀ aryl, unsubstituted or substituted benzyl, unsubstituted or substituted phenyl, unsubstituted or substituted benzylcarbonyl, unsubstituted or substituted phenylcarbonyl, or mono- or polysaccharides. Preferred mono- and polysaccharides include ribose, glucose, deoxyribose, dextran, starch, glycogen, lactose, fucose, galactose, fructose, glucosamine, galactosamine, heparin, mannose, maltose, sucrose, sialic acid, cellulose, and combinations thereof.

All moieties of Q₁, Q₂, and Q₃, other than hydrogen, can be optionally substituted with 1 to 5 substituents, where the substituents can be the same or different. Exemplary substituents for Q₁₋₃ include nitro, halo, hydroxy, C₁₋₂₄ alkyl, C₁₋₂₄ alkoxy, amino, mono-C₁₋₂₄ alkylamino, di-C₁₋₂₄ alkylamino, cyano, phenyl and phenoxy. Also, Y can be optionally substituted. Exemplary substituents for Y include unsubstituted or substituted C₁₋₂₄ aliphatic polyamines, unsubstituted or substituted C₃₋₂₄ cycloalkylamines, unsubstituted or substituted C₃₋₂₄ heterocycloalkylamines, unsubstituted or substituted C₃₋₃₀ arylamines, such as unsubstituted or substituted phenyl amines, unsubstituted or substituted benzylamines, unsubstituted or substituted benzylamine carbonyls, unsubstituted or substituted phenylamine carbonyls, and combinations thereof.

Exemplary amine-functionalized silanes encompassed within the scope of the invention include 3-aminopropyltrimethoxysilane, 3-aminopropyltriethoxysilane, 3-aminopropyldimethoxysilane, N-(3-acryloxy-2-hydroxypropyl)-3-amino-propyltriethoxysilane, N-2-(aminoethyl)-3-aminopropyltris(2-ethyl-hexoxy)silane, 3-(m-aminophenoxy)propyltrimethoxysilane, 3-(1-aminopropoxy)-3,3-dimethyl-1-propenyl-trimethyoxysilane, 3-aminopropyltris(methoxyethoxyethoxy)silane, 3-aminopropylmethyldiethoxysilane, 3-aminopropyltris(trimethylsiloxy)silane, bis(dimethylamino)methylchlorosilane, bis(dimethylamino)methylmethoxysilane, bis(dimethylamino)phenylchlorosilane, bis(dimethylamino)phenylethoxysilane, bis(2-hydroxyethyl)-3-aminopropyltriethoxysilane, bis(2-hydroxyethyl)-3-aminopropyltrimethoxysilane, bis(3-triethoxysilyl)propylamine, 1,4-bis[3-(trimethoxysilyl)propyl]ethylenediamine, (N,N-diethyl-3-aminopropyl)trimethoxysilane, (N,N-dimethyl-3-aminopropyl)trimethoxysilane, N-phenylaminopropyltrimethoxysilane, trimethoxysilylpropyldiethylenetriamine, trimethoxysilylpropylpentaethylenehexamine, triethoxysilyloctyldiethylenetriamine, triisopropoxysilylpentaethylenehexamine, 3-aminopropylmethyldiethoxysilane, 2-(perfluorooctyl)ethyltriaminotrimethoxysilane, 4-aminobutyltrimethoxysilane, N-(6-aminohexyl)aminopropyltrimethoxysilane, 3-(dimethoxymethylsilylpropyl)diethylenetriamine, N-(2-aminoethyl)-N′-[3-(dimethoxymethylsilyl)propyl]-1,2-ethanediamine, amine-functionalized polydimethylsiloxane copolymer (available from Dow Corning as “MDX4-4159”), and combinations thereof. The amine-functionalized silane compounds also include bis-aminosilanes such as, for example, bis-(trimethoxysilylpropyl)amine, bis-(triethoxysilylpropyl)amine, bis-(triethoxysilylpropyl)ethylene diamine, N-[2-vinylbenzylamino)ethyl]-3-aminopropyltrimethoxysilane, aminoethylaminopropyltrimethoxysilane, trimethoxysilyl-modified polyethylenimine, methyldimethoxysilyl-modified polyethylenimine, and combinations thereof. Other exemplary amine-functionalized silanes include those disclosed and described in, for example, PCT Application No. WO 00/63462, and are incorporated by reference.

The amine-functionalized silanes can be used alone or in combination with one another. Additionally, the amine-functionalized silanes of the invention can be used as a mixture with other mono-, oligo-, or polymeric functionalized and nonfunctionalized silanes and silicones, such as, for example, 2-acetoxyethyltrichlorosilane, 2-acetoxyethyldimethylchlorosilane, acryloxypropylmethyldimethoxysilane, 3-acryloxypropyltrichlorosilane, 3-acryloxypropyltrimethoxysilane, adamantylethyltrichlorosilane, allyldimethylchlorosilane, allyltrichlorosilane, allyltriethoxysilane, allytrimethoxysilane, amyltrichlorosilane, amyltriethoxysilane, amyltrimethoxysilane, 5-(bicycloheptenyl)methyldichlorosilane, 5-(bicycloheptenyl)methyltriethoxysilane, 5-(bicycloheptenyl)methyltrimethoxysilane, 5-(bicycloheptenyl)dimethylmethoxysilane, 5-(bicycloheptenyl)methyldiethoxysilane, bis(3-cyanopropyl)dichlorosilane, bis(3-cyanopropyl)diethoxysilane, bis(3-cyanopropyl)dimethoxysilane, 1,6-bis(trimethoxysilyl)hexane, bis(trimethylsiloxy)methylsilane, bromomethyldimethylchlorosilane, bromomethyldimethylmethoxysilane, 3-bromopropyltrichlorosilane, 3-bromopropyltriethoxysilane, n-butyldimethylchlorosilane, n-butyldimethylmethoxysilane, tert-butyldimethylchlorosilane, ter-butyldimethylisoproplysilane, tert-butyldiphenylchlorosilane, tert-diphenylmethoxysilane, n-butylmethyldichlorosilane, n-butyldimethoxysilane, n-butyldiethoxysilane, n-butyldiisopropylsilane, n-butyltrimethoxysilane, (10-carbomethoxydecyl)dimethylchlorosilane, 2-(carbomethoxy)ethyltrimethoxysilane, 4-chlorobutyldimethylmethoxysilane, 4-chlorobutyldimethylethoxysilane, 2-chloroethylmethyldiisopropylsilane, 2-chloroethyltriethoxysilane, chloromethyldimethylethoxysilane, p-(chloromethyl)phenyltriethoxysilane, p-(chloromethyl)phenyltrimethoxysilane, chloromethyltriethoxysilane, chlorophenyltrimethoxysilane, 3-chloropropylmethyldimethoxysilane, 3-chloropropyltriethoxysilane, 2-(4-chlorosulfonylphenyl)ethyltrichlorosilane, 2-cyanoethylmethyltrimethoxysilane, (cyanomethylphenethyl)triethoxysilane, 3-cyanopropyldimethyldiisopropylsilane, 2-(3-cyclohexenyl)ethyl]trimethoxysilane, cyclohexydiethoxymethylsilane, cyclopentyltrimethoxysilane, di-t-butoxydiacetoxysilane, di-n-butyldimethoxysilane, dicyclopentyldimethoxysilane, diethyldiethoxysilane, diethyldimethoxysilane, diethyldibutoxysilane, diethylphophatoethyltriethoxysilane, diethyl(triethoxysilylpropyl)malonate, di-n-hexyldimethoxysilane, diisopropyldichlorosilane, diisopropyldimethoxysilane, dimethyldiacetoxysilane, dimethyldimethoxysilane, 2,3-dimethylpropyldimethylethoxysilane, dimethylethoxysilane, dimethylmethoxychlorosilane, dimethyl-n-octadecylchlorsilane, N,N-dimethyltriethylsilylamine, 1,3-diemethyltetramethoxydisoloxane, diphenylchlorosilane, diphenyldiacetoxysilane, diphenydiethoxysilane, diphenyldifluorosilane, diphenyldimethoxysilane, diphenylmethylchlorosilane, diphenylmethylethoxysilane, 2-(diphenylphosphino)ethyltriethoxysilane, divinylethoxysilane, divinyldichlorosilane, n-docosylmethyldichlorosilane, n-dodecyltriethoxysilane, 2-(3,4-epoxycyclohexyl)ethyltrimethoxysilane, ethyldimethylchlorosilane, ethyltriacetoxysilane, ethyltriethoxysilane, ethyltrimethoxysilane, 3-glycidoxypropyldimethylethoxysilane, (3-glycidoxypropyl)methyldimethoxysilane, 3-glycidoxypropyltrimethoxysilane, (3-heptafluoroisopropoxy)propylmethyldichlorosilane, n-heptylmethyldichlorosilane, n-heptylmethyldimethoxysilane, n-hexadecyltrichlorosilane, n-hexadecyltriethoxysilane, 6-hex-1-enyltrichlorosilane, 5-hexenyltrimethoxysilane, n-hexylmethyldichlorosilane, n-hexyltrichlorosilane, n-hexytriethoxysilane, n-hexyltrimethoxysilane, 3-iodopropyltriethoxysilane, 3-iodopropyltrimethoxysilane, isobutyldimethylchlorosilane, isobutylmethyldichlorosilane, isobutyltrimethoxysilane, isobutyltriethoxysilane, 3-isocyanatopropyldimethylchlorosilane, isocyanatopropyldimethylmethoxysilane, 3-isocyanatopropyltriethoxysilane, isooctyltrichlorsilane, isooctyltriethoxysilane, isopropyldimethylchlorosilane, 3-mercaptopropylmethyldimethoxysilane, 3-mercaptopropyltrimethoxysilane, 3-mercaptopropyltriethoxysilane, 3-methacryloxypropylmethyldiethoxysilane, 3-methacryloxypropyl-methyldimethoxysilane, 3-methacryloxypropyltrimethoxysilane, 3-(4-methoxyphenyl)propyltrichlorosilane, 3-(4-methoxyphenyl)propyltrimethoxysilane, methylcyclohexyldichlorosilane, methylcyclohexyldiethoxysilane, methyldiacetoxysilane, methyldichlorosilane, methyldiethoxysilane, methyldimethoxysilane, methyldodecyldichlorosilane, methyldodecyldiethoxysilane, methylisopropyldichlorosilane, methyl-n-octadecyldimethoxysilane, methyl-n-octyldichlorosilane, (p-methylphenethyl)methyldichlorosilane, methyl(2-phenethyl)dimethoxysilane, methylphenyldiisopropoxysilane, methylphenyldiethoxysilane, methylphenyldimethoxysilane, methyl-n-propyldimethoxysilane, methyltriacetoxysilane, methyltriethoxysilane, neophylmethyldiethoxysilane, n-octadecyldimethylmethoxysilane, n-octadecyltriethoxysilane, n-octadecyltrimethoxysilane, 7-oct-1-enylmethylchlorosilane, 7-oct-enyltrimethoxysilane, n-octyldiisopropylchlorosilane, n-octyldimethylchlorosilane, n-octylmethyldimethoxysilane, n-octyltriethoxysilane, 1,1,1,3,3-pentamethyl-3-acetoxydisiloxane, phenethyldimethylchlorosilane, phenethyldimethylmethoxysilane, phenethyltriethoxysilane, phenyl(3-chloropropyl)dichlorosilane, phenyldimethylacetoxysilane, phenyldimethylethoxysilane, phenylmethylvinylchlorosilane, (3-phenylpropyl)dimethylchlorosilane, phenyltriethoxysilane, phenyltrimethoxysilane, phthalocyanatodimethoxysilane, n-propyldimethylchlorosilane, n-propyltrimethoxysilane, styrylethyltrimethoxysilane, tetra-n-butoxysilane, tetraethoxysilane, tetramethoxysilane, tetraproproxysilane, (tridecafluoro-1,1,2,2,-tretrahydrooctyl)-1-trimethoxysilane, triethoxysilane, triethoxysilylpropylethyl carbamate, triethylacetoxysilane, triethylethoxysilane, (3,3,3-trifluoropropyl)dimethylchlorosilane, (3,3,3-trifluoropropyl)methyldimethoxysilane, (3,3,3-trifluoropropyl)triethoxysilane, triisopropylchlorosilane, trimethoxysilane, 1-trimethoxysilyl-2-(p,m-chloromethyl)-phenylethane, trimethylethoxysilane, 2-(trimethylsiloxy)ethyl methacrylate, p-trimethylsiloxynitrobenzene, o-trimethylsilylacetate, triphenylethoxysilane, n-undeceyltrimethoxysilane, vinyldimethylethoxysilane, vinyltriacetoxysilane, vinyltrimethoxysilane, and combinations thereof. Optionally, substrates can be alternatively or successively coated with amine-functionalized and functionalized/nonfunctionalized silanes and silicones. Additional functionalized and nonfunctionalized silanes and silicones encompassed within the scope of the invention include those disclosed and described in, for example, United Chemical Technologies, Inc. Catalog CD (1999-2000), and are incorporated herein by reference.

Preferably, the substrate is cleaned according to procedures well known in the art prior to reaction with the silane reagent(s). To prepare the nitric oxide-releasing coated substrates of the invention, the substrate (e.g., stainless steel) is contacted with a composition containing an amine-functionalized silane compound or oligomer thereof. The amine-functionalized silane is preferably hydrolyzed in the presence of a hydrolyzing reagent. The hydrolyzing reagent can be any reagent capable of hydrolyzing the silane.

The amine-functionalized silane compound is preferably hydrolyzed prior to contacting it with the substrate. More preferably, the amine-functionalized silane compound is dissolved, suspended, dispersed, or the like in a composition comprising a hydrolyzing reagent. Most preferably, the amine-functionalized silane compound is dissolved in a composition comprising a hydrolyzing reagent. The hydrolyzing reagent hydrolyzes the silane to form mono- and oligomeric silane. Advantageously, therefore, one or more silanes are dissolved in the hydrolyzing reagent, such as water, or solvent comprising the hydrolyzing reagent containing at least one molar equivalent of water to facilitate its hydrolysis such that oligomer formation is the predominant reaction. Preferable solvents for this transformation include those known in the art, such as, for example, methanol, ethanol, isopropanol, tetrahydrofuran, acetonitrile, and the like that are readily miscible with water. Optionally, however, the amine-functionalized silane compound can be mixed in a silicone gel containing at least one molar equivalent of water and applied to the substrate.

The amine-functionalized silane compositions or solutions are contacted with the substrate using methods known in the art including, for example, dipping, spraying, brushing, imbibing, and rolling. While not wishing to be bound to any particular theory, it is believed that after the amine-functionalized oligomeric silane composition is contacted with the substrate, functional groups (e.g., hydroxyls) on the surface of the substrate react with the silane derivatives to form covalent bonds between silane and the substrate. Preferably, the silane-coated substrate is cured. Curing can occur at any temperature, pressure, or in the presence or absence of an inert gas/gas mixture, in the presence of absence of moisture, or an external energy source, such as heat or other radiation, e.g., gamma radiation, or mechanical energy, e.g., sonic energy, so long as the amine-functionalized polysilane layers formed during this step are not damaged, i.e., rendering them incapable of further reiterative coating cycles and/or diazeniumdiolation with NO. It is particularly preferred to cure the substrate under conditions that will preserve the nucleophile residue groups so that such groups are available for diazeniumdiolation. The number of such coating and curing cycles may be repeated to any desired level, so as to optimize the amount and period of NO released from the coated substrate.

The nitric oxide-releasing functional group is any suitable group capable of releasing NO. The nitric oxide-releasing functional group is preferably a diazeniumdiolated nitric oxide-releasing/nucleophile residue, i.e., a complex of nitric oxide and a nucleophile, most preferably a nitric oxide/nucleophile residue complex which contains the anionic moiety X [symbol] N(O)NO]⁻, X [symbol] N(O)NO [symbol] X or X—NO, where X is any suitable nucleophile residue. Preferably, nitric oxide-releasing functional groups of the invention are formed according to the following formula
X—+2NO→X [symbol] N(O)NO]—

The nucleophile residue is most preferably that of a primary amine (e.g., X═(CH₃)₂CHNH, as in (CH₃)₂CHNH[N(O)NO]Na), a secondary amine (e.g., X═(CH₃CH₂)₂N, as in (CH₃CH₂)₂N[N(O)NO]Na), a polyamine (e.g., X=spermine, as in the zwitterions H₂N(CH₂)₃NH₂⁺(CH₂)₄N[N(O)NO]⁻(CH₂)₃NH₂, X=(ethylamino)ethylamine, as in the zwitterion CH₃CH₂N[N(O)NO]⁻CH₂CH₂NH₃⁺, X=3-(n-propylamino)propylamine, as in the zwitterion CH₃CH₂CH₂N[N(O)NO]⁻CH₂CH₂CH₂NH₃⁺), oxide (i.e., X═O⁻, as in Na₂O[N(O)NO]), or derivatives thereof. Such nitric oxide/nucleophile residue complexes are stable as solids and are capable of releasing nitric oxide in a biologically useful form at a predictable rate. Most preferably, the nitric oxide/nucleophile residue complexes of the present invention are formed from a hydrolyzable amine-functionalized organosilane moiety. Suitable nitric oxide/amine-functionalized organosilanes include those described herein, wherein Q₂ is [N(O)NO]⁻ Q₂ or Q₃ is [N(O)NO [symbol] X; optionally, each Q₂ and Q₃ are the same or different, hydrogen, unsubstituted or substituted C₁₋₂₄ aliphatic, unsubstituted or substituted C₃₋₁₂ olefinic, unsubstituted or substituted C₃₋₂₄ cycloalkyl, unsubstituted or substituted C₃₋₂₄ heterocycloalkyl, unsubstituted or substituted C₃₋₃₀ aryl, unsubstituted or substituted benzyl, unsubstituted or substituted phenyl, unsubstituted or substituted benzylcarbonyl, unsubstituted or substituted phenylcarbonyl, or saccharides. Preferred saccharides include ribose, glucose, deoxyribose, dextran, starch, glycogen, lactose, fucose, galactose, fructose, glucosamine, galactosamine, heparin, mannose, maltose, sucrose, sialic acid and cellulose.

Other suitable nitric oxide/nucleophile residue complexes that can provide the NO-releasing functional group are well known in the art and include, for example, those described in U.S. Pat. Nos. 4,954,526, 5,039,705, 5,155,137, 5,121,204, 5,250,550, 5,366,997, 5,405,919, 5,525,357 and 5,650,447 to Keefer et al. and in Hrabie et al., J. Org. Chem. 58: 1472-1476 (1993), and are incorporated herein by reference.

Exemplary nitric oxide/nucleophile residue complexes that can provide the NO-releasing functional group include those having the following formulas: 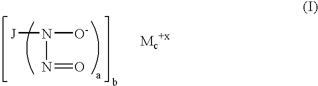
wherein J is an organic or inorganic moiety, including, for example, a moiety which is not linked to the nitrogen of the N₂O₂⁻ group through a nitrogen atom, M^{+x} is a pharmaceutically acceptable cation, where x is the valence of the cation, a is 1 or 2, and b and c are the smallest integers that result in a neutral compound, preferably such that the compound is not a salt of alanosine or dopastin, as described in U.S. Pat. No. 5,212,204, and are incorporated herein by reference; 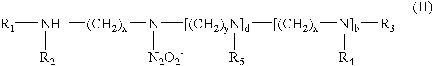
wherein b and d are the same or different and may be zero or one, R₁, R₂, R₃, R₄, and R₅ are the same or different and may be hydrogen, C₃₋₈ cycloalkyl, C₁₋₁₂ straight or branched chain alkyl, benzyl, benzoyl, phthaloyl, acetyl, trifluoroacetyl, p-toluyl, t-butoxycarbonyl, or 2,2,2-trichloro-t-butoxycarbonyl, and x, y, and z are the same or different and are integers from 2 to 12, as described in U.S. Pat. No. 5,155,137, and are incorporated herein by reference; 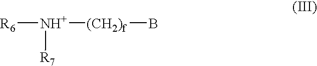
wherein B is 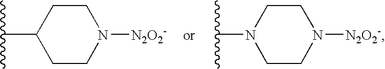
R₆ and R₇ are the same or different and are hydrogen, C₃₋₈ cycloalkyl, C₁₋₁₂ linear alkyl, or C₃₋₁₂ branched alkyl, or benzyl. Alternatively, compounds of formula (III) do not comprise a H⁺ moiety associated with the nitrogen when R₆ and R₇ are the same or different and are benzoyl, phthaloyl, acetyl, trifluoroacetyl, p-toluyl, t-butoxycarbonyl, or 2,2,2-trichloro-t-butoxycarbonyl. In addition, f is an integer from 0 to 12, with the proviso that when B is the substituted piperazine moiety 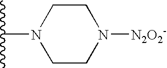
then f is an integer from 2 to 12, as described in U.S. Pat. No. 5,155,137, and are incorporated herein by reference; 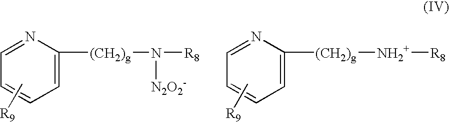
wherein R₈ is hydrogen, C₃₋₈ cycloalkyl, C₁₋₁₂ linear alkyl, C₃₋₁₂ branched alkyl, or benzyl. Compounds of formula (IV) do not comprise a H⁺ moiety associated with the nitrogen when R₈ is benzoyl, phthaloyl, acetyl, trifluoroacetyl, p-toluyl, t-butoxycarbonyl, or 2,2,2-tri-chloro-t-butoxycarbonyl. R₉ is hydrogen or a C₁₋₁₂ linear alkyl, C₃₋₁₂ branched alkyl, and g is 2 to 6, as described in U.S. Pat. No. 5,250,550, and are incorporated herein by reference; 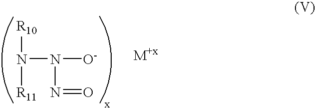
wherein R₁₀ and R₁₁ are independently selected from the group consisting of a linear C₁₋₁₂ alkyl or C₃₋₁₂ branched alkyl group and a benzyl group, preferably such that no branch occurs on the alpha carbon atom, or else R₁₀ and R₁₁, together with the nitrogen atom to which they are bonded, to form a heterocyclic group, preferably a pyrrolidino, piperidino, piperazino or morpholino group, M^{+x} is a pharmaceutically acceptable cation, and x is an integer from 1 to 10, as described in U.S. Pat. Nos. 5,039,705, 5,208,233 and 5,731,305, and are incorporated herein by reference;
K[(M)^{x′}ₓ(L)_{y}(R₁₂R₁₃N—N₂O₂)_{z}] (VI)
wherein M is a pharmaceutically acceptable metal, or, where x is at least two, a mixture of two different pharmaceutically acceptable metals, L is a ligand different from (R₁₂R₁₃N—N₂O₂) and is bound to at least one metal, R₁₂ and R₁₃ are each organic moieties and may be the same or different, x is an integer of from 1 to 10, x′ is the formal oxidation state of the metal M, and is an integer of from 1 to 6, y is an integer of from 1 to 18, and where y is at least 2, the ligands L may be the same or different, z is an integer of from 1 to 20, and K is a pharmaceutically acceptable counterion to render the compound neutral to the extent necessary, as described in U.S. Pat. No. 5,389,675, and are incorporated herein by reference;
[R₁₄N(H)N(NO)O⁻]_{y}X (VII)
wherein R₁₄ is C₂₋₈ alkyl, phenyl, benzyl, or C₃₋₈ cycloalkyl, any of which R₁₄ groups may be substituted by 1 to 3 substituents, which are the same or different, selected from the group consisting of halo, hydroxy, C₁₋₈ alkoxy, —NH₂, —C(O)NH₂, —CH(O), —C(O)OH, and —NO₂, X is a pharmaceutically acceptable cation, a pharmaceutically acceptable metal center, or a pharmaceutically acceptable organic group selected from the group consisting of C₁₋₈ alkyl, —C(O)CH₃, and —C(O)NH₂, and y is one to three, consistent with the valence of X, as described in U.S. Pat. No. 4,954,526, and are incorporated herein by reference; and 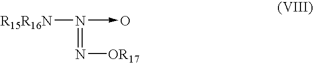
wherein R₁₅ and R₁₆ are independently chosen from C₁₋₁₂ linear alkyl, C₁₋₁₂ alkoxy or acyloxy substituted straight chain alkyl, C₂₋₁₂ hydroxy or halo substituted straight chain alkyl, C₃₋₁₂ branched chain alkyl, C₃₋₁₂ hydroxy, halo, alkoxy, or acyloxy substituted branched chain alkyl, C₃₋₁₂ linear alkenyl, and C₃₋₁₂ branched alkenyl which are unsubstituted or substituted with hydroxy, alkoxy, acyloxy, halo or benzyl, or R₁₅ and R₁₆, together with the nitrogen atom to which they are bonded, form a heterocyclic group, preferably a pyrrolidino, piperidino, piperazino or morpholino group, and R₁₇ is a group selected from C₁₋₁₂ linear and C₃₋₁₂ branched alkyl which are unsubstituted or substituted by hydroxy, halo, acyloxy or alkoxy, C₂₋₁₂ linear or C₃₋₁₂ branched alkenyl which are unsubstituted or substituted by halo, alkoxy, acyloxy or hydroxy, C₁₋₁₂ unsubstituted or substituted acyl, sulfonyl and carboxamido; or R₁₇ is a group of the formula —(CH₂)ₙ—ON═N(O)NR₁₅R₁₆, wherein n is an integer of 2-8, and R₁₅ and R₁₆ are as defined above. Preferably R₁₅, R₁₆, and R₁₇ do not contain a halo or a hydroxy substituent alpha to a heteroatom, as described in U.S. Pat. No. 5,366,997, and are incorporated herein by reference.

Preferably, the nitric oxide-releasing functional group is at least one compound consisting of an O²-protected monodiazeniumdiolate of piperazine, such as the O²-glycosylated or methoxymethyl-protected monodiazeniumdiolate of piperazine. Another preferred nitric oxide-releasing functional group is a 1-[(2-carboxylato)pyrrolidin-1-yl]diazen-1-ium-1,2-diolate because the metabolite of the nitric oxide-releasing functional group is proline, an amino acid.

Other preferred nitric oxide/nucleophile residue complexes that can provide the NO-releasing functional group include O²-arylated and O²-glycosylated diazeniumdiolates, such as those described in the international patent application PCT/US97/17267 (filed Sep. 26, 1997), and are incorporated herein by reference. For example, a preferred O²-aryl substituted diazeniumdiolate has the following formula 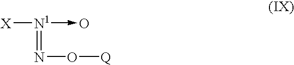
wherein X is selected from the group consisting of an amino, a polyamino, a C₁₋₂₄ aliphatic, a C₃₋₃₀ aryl, a C₃₋₃₀ nonaromatic cyclic, an oxime, a polycyclic, and an aromatic polycyclic, and Q is an aryl group selected from the group consisting of an acridine, an anthracene, a benzene, a benzofuran, a benzothiophene, a benzoxazole, a benzopyrazole, a benzothiazole, a carbazole, a chlorophyll, a cinnoline, a furan, an imidazole, an indole, an isobenzofuran, an isoindole, an isoxazole, an isothiazole, an isoquinoline, a naphthalene, an oxazole, a phenanthrene, a phenanthridine, a phenothiazine, a phenoxazine, a phthalimide, a phthalazine, a phthalocyanine, a porphin, a pteridine, a purine, a pyrazine, a pyrazole, a pyridazine, a pyridine, a pyrimidine, a pyrrocoline, a pyrrole, a quinolizinium ion, a quinoline, a quinoxaline, a quinazoline, a sydnone, a tetrazole, a thiazole, a thiophene, a thyroxine, a triazine, and a triazole, wherein an atom of the ring of the aryl group is bonded to the O²-oxygen.

With respect to O²-glycosylated diazeniumdiolates, a preferred embodiment includes an O²-glycosylated 1-substituted diazen-1-ium-1,2-diolate of Formula IX. Preferably, X is selected from the group consisting of an amino, a polyamino, a C₁₋₂₄ aliphatic, a C₃₋₃₀ aryl and a C₃₋₃₀ non-aromatic cyclic, and Q is a saccharide. Optionally, Q is a protecting group, such as those known in the art (See, e.g., Greene et al., “Protecting Groups In Organic Synthesis,” J. Wiley & Sons: New York, 1999, and are incorporated herein by reference). Most preferably, the O²-substituted diazeniumdiolate includes an O²-substituted 1-[(2-carboxylato)pyrrolidin-1-yl]diazen-1-ium- 1,2-diolate.

Other preferred nitric oxide/nucleophile residue complexes that can provide the NO-releasing functional group include enamine- and amidine-derived diazeniumdiolates, such as those described in the international patent publication No. WO 99/01427 (PCT/US98/13723), and are incorporated herein by reference.

The nitric oxide-releasing functional group may also be that of a polymer, e.g., a nitric oxide-releasing/nucleophile complex bound to a polymer such as those described in, for example, U.S. Pat. Nos. 5,405,919, 5,525,357, 5,632,981, 5,650,447, 5,676,963, 5,691,423, and 5,718,892, and are incorporated herein by reference. By “bound to a polymer” it is meant that the nitric oxide-releasing/nucleophile complex, such as those described by Formulae I-IX is associated with, part of, incorporated with, or contained within the polymer matrix physically or chemically. Physical association or bonding of the nitric oxide-releasing/nucleophile complex to the polymer may be achieved by co-precipitation of the polymer with the nitric oxide-releasing/nucleophile complex as well as by covalent bonding of the complex to the polymer. Chemical bonding of the nitric oxide-releasing/nucleophile complex to the polymer may be by, for example, covalent bonding of the nucleophile residue moiety of the nitric oxide-releasing/nucleophile complex to the polymer such that the nucleophile to which the NONO group is attached forms part of the polymer itself, i.e., is in the polymer backbone, or is attached to groups pendant to the polymer backbone. The manner in which the nitric oxide-releasing/nucleophile complex is associated, part of, or incorporated with or contained within, i.e., “bound” to the polymer, is inconsequential to the invention and all means of association, incorporation or bonding are contemplated herein. Preferably the nitric oxide-releasing/nucleophile complex is covalently bound to the polymer.

The nucleophile residue is preferably an amine-derived residue, e.g., primary or secondary amines, such as those described herein. The amine-derived nucleophile residue(s) is preferably a diethylenetriamine, pentaethylenehexamine, high molecular weight linear/branched polyethylenimines, polyamine-functionalized divinylbenzene, piperazine, or any combination thereof.

It has been found that substrates coated with amine-functionalized silanes in accordance with the invention were found to be sufficiently stable to (i) allow for diazeniumdiolation with NO and (ii) spontaneously release NO under physiological conditions. These unexpected results permit the development of medical devices, such as those described herein that are capable of sustained NO-release in accordance with the teachings of the invention.

The substrates can be converted into diazeniumdiolates once they have been provided with an amine-functionalized polysilane coating in accordance with the teachings of the invention. Briefly, the nitric oxide-releasing substrates of the invention are formed by contacting the previously processed substrates (reiteratively coated amine-functionalized silylated substrate) with nitric oxide or a nitric oxide-releasing functional group. Alternatively, the substrates can be converted into diazeniumdiolates once they have been provided with a nucleophile residue by contacting the nucleophile residue with NO gas either neat or, preferably, in a suitable solvent or solvent mixture.

Combinations of direct diazeniumdiolation and bonding of nitric oxide-releasing functional group are also within the scope of the invention.

In one preferred embodiment, the degree of diazeniumdiolation is controlled by the solvent system used to form the diazeniumdiolated amine-functionalized salts. Without being bound to any particular theory, it is believed that when the amine-functionalized polysilane coated substrate is treated with NO in a pure organic solvent such as acetonitrile, every other amine group in the polymeric coating may be converted to a diazeniumdiolate group. The nonderivatized amine groups of the polymer are believed to form ammonium cations resulting overall in a stable zwitterionic salt. However, in another preferred embodiment, when a solvent containing an organic or mineral base such as, for example, ammonium hydroxide, triethylamine, sodium methylate or sodium trimethylsilanoate are used, diazeniumdiolate groups can, in principle, be formed on every available secondary amine resulting in stable diazeniumdiolated organic or mineral salts of the polymer. Similarly, other organic or mineral bases also yield the corresponding diazeniumdiolated organic or mineral salts, such as those of tetrabutylammonium, dimethylethylammonium, potassium, calcium, silver, magnesium and the like.

If desired, before diazeniumdiolation with NO gas, the amine-functionalized polysilane coated substrate can be treated with a bio- or hemocompatible topcoat. The topcoat is any suitable lubricious hydrogel. Suitable lubricious hydrogels include, for example, hydrophilic silicones, homo- and heteropolyethers, polyols, polyureas, polylactones, perfluorinated hydrocarbons, albumin-, heparin-, and phosphorylcholine-functionalized polymers, or any combination thereof.

Another preferred embodiment of the invention is forming a hydrophobic topcoat on the amine-functionalized polysilane coated substrate. Suitable hydrophobic topcoats include, for example, unsubstituted and substituted parylenes, unsubstituted and substituted polydivinylbenzenes, unsubstituted and substituted polysiloxanes, silicones and the like.

A further embodiment of this invention includes forming successive layers of different amine-functionalized polysilanes. After a first amine-functionalized silane is bound to the substrate, at least one additional amine-functionalized silane that is the same or different is bonded to the first layer. This procedure may be reiterated as often as deemed necessary to increase the number of bonding sites capable of being diazeniumdiolated with NO. It follows that the greater the number of bonding sites capable of being diazeniumdiolated with NO, the greater the amount of NO will be released under appropriate conditions. Alternatively, the reiteratively layered amine-functionalized coatings of the present invention can be reacted with a nitric oxide-releasing functional group (e.g., anionic diazeniumdiolates) or an anionic compound (e.g., L-proline) to form organic salt complexes. Upon exposure to NO then physiological solutions, these coatings will also release NO and/or the anionic component of the complex. The formation of combinations of direct diazeniumdiolation and bonding of nitric oxide-releasing functional groups is also within the scope of the invention.

A further embodiment of this invention includes mixing or forming an amine-functionalized polysilane without a substrate present in order to produce a nitric oxide-releasing material such as, for example, an NO-releasing silicone rubber. A first hydrolyzable amine-functionalized silane is contacted with an additive or optionally, with at least one additional hydrolyzable functionalized or nonfunctionalized silane, so as to form a polysilane-based material. The additional hydrolyzable functionalized silane(s) can be the same or different than the first hydrolyzable amine-functionalized silane. The additive can be any suitable material that induces a desired property of the resulting material. For example, the addition of boric acid is known in the art to produce silanes with improved elasticity. Other such additives are well known in the art. See, e.g., Brook, M. A., “Silicon in Organic, Organometallic, and Polymer Chemistry” (J. Wiley & Sons: New York, 1999); “The Chemistry of Organic Silicon Compounds. Vol. 2, Parts 1, 2 and 3,” Rappaport, Z., Apeloig, Y., Eds. (J. Wiley & Sons: New York, 1998), the entire contents of which are incorporated herein by reference.

Yet another embodiment of the invention provides a substrate, such as a medical device, for delivering nitric oxide in therapeutic concentrations for a sustained period of time. The substrate includes a polysilane coating comprising at least one amine-functionalized silane, and having nitric oxide releasably bound thereto, such as in the form of diazeniumdiolated nucleophile residues. The polysilane coating is reiteratively layered, and the amine-functionalized silanes can be the same or different.

The resulting diazeniumdiolated substrates in accordance with the invention can be tested to determine the concentration and duration of NO release upon exposure to physiological conditions by methods known in the art (e.g., immersion in phosphate buffered saline, pH 7.4 at 37° C.). Nitric oxide gas is preferably detected and quantified using the chemiluminescence methods as described in Keefer et al., “NONOates (1-Substituted Diazen-1-ium-1,2 diolates) as Nitric Oxide Donors: Convenient Nitric Oxide Dosage Forms,” Methods in Enzymology 28: 281-293 (1996), and are incorporated herein by reference.

The NO-releasing substrates of the invention have been found to generate between about 1,000 to about 40,000 pmoles per square millimeter (mm²) of coated substrate, more particularly between about 2,000 to about 35,000 pmoles per square millimeter (mm²), more particularly between about 5,000 to about 20,000 pmoles per square millimeter (mm²), and even more particularly between about 8,000 to about 13,000 pmoles per square millimeter (mm²). However, both the yield and duration of NO can be readily increased by coating the substrates with additional layers of the amine-functionalized polysilanes per the teachings of the invention. Moreover, the NO-releasing substrates of the invention can continually release NO for periods of hours to weeks or even longer. These findings far exceed those of any previously reported amine-functionalized polysilane coating in terms of the amounts or duration of NO released.

The reiteratively layered substrates of the invention provide localized release of nitric oxide under physiological conditions. The localized release or localized sustained release of NO provides in situ cytostatic, antithrombogenic, vasodilatory, antiproliferative, and other pharmacological effects. The NO-releasing substrates of the invention are thromboresistant when in contact with blood and are capable of inhibiting arterial restenosis as well promoting angiogenesis. Accordingly, when used alone, as a coating on, or in combination with, other substances (e.g., stainless steel, glass, silicone rubber, plastics, natural fibrous materials, etc.) many uses are contemplated.

The NO-releasing substrates of the invention can be used to treat or prevent a wide range of conditions including, for example, ischemic heart disease, restenosis, cancer, hypertension, infectious diseases, and sexual dysfunction. Potential commercial applications include, for example, the preparation of coated NO-releasing medical devices, as defined herein, including stents, surgical/dental devices, catheters, syringes, needles, blood collection tubes and bags, disposable contact lenses, prostheses, implants, pacemakers, pacemaker leads, heart valves, pulse generators, cardiac defibrillators, cardioverter defibrillators, spinal stimulators, brain and nerve stimulators, introducers, chemical sensors, artificial joints, skin/vascular grafts, bandages and dressings, chemical and physiological electrodes/sensors, personal hygiene and contraceptive items. Optionally, the amine-functionalized polysilane coatings of the present invention can also be used to bind and selectively deliver drugs, prodrugs, nucleotides, oligonucleotides, polynucleotides, amino acids, proteins, saccharides as well as fix tissue slices/specimens for histological or pathological examination, and the like, according to methods known in the art.

The following examples further illustrate the invention but, of course, should not be construed as in any way limiting its scope.

### EXAMPLE 1

This Example illustrates the preparation of a reiteratively coated medical device. Trimethoxysilylpropyldiethylenetriamine (2.5 g), methanol (7.125 g) and deionized water (0.375 g) were added to a small vial. The solution was mixed for several minutes using a roller mixer and transferred to an airbrush container. An isopropanol-cleaned 1×6×0.05 cm stainless steel coupon was attached to a SLO-SYN motor (200 RPM). The stainless steel coupon was subjected to the following procedure: sprayed for 3 seconds, rotated in air for 15 seconds, sprayed for 3 seconds, rotated in air for 15 seconds, sprayed for 3 seconds, and rotated in air for 15 seconds. The coupon was then placed in an oven at 60° C. to cure for 30 minutes. After the coupon was removed from the oven and allowed to cool to room temperature, the procedure was repeated two additional times. The reiteratively- or multiply-coated coupon was placed in an oven at 60° C. overnight to cure.

The next morning, the coupon was removed from the oven and allowed to cool to room temperature. Next, the coupon was placed in a test tube immersed in acetonitrile. The tube was then transferred to a Parr® hydrogenation pressure vessel and oxygen was removed from the vessel using repeated cycles of pressurization/depressurization with nitrogen gas. This was followed by the introduction of NO at a pressure of 276 kPa (40 psi). The tube containing the coupon was exposed to the NO gas for 24 hours. The acetonitrile was decanted, the coupon was washed with 20 mL of diethyl ether, and flushed with nitrogen gas until dry. The NO content of the coupon was determined by immersing an approximately 1×1×0.05 cm piece of the diazeniumdiolated coupon in 0.1 M phosphate buffer, pH 7.4 at 37° C., whereupon chemiluminescence-detectable NO was evolved over approximately a 10 day period of analysis. The total NO release was measured at 10,060 pmoles/mm².

### EXAMPLE 2

This Example illustrates the preparation of a nitric oxide-releasing substituted ammonium salt of a mixed diethylenetriaminopropylpolysilane and dimethylpolysilane-coated stainless steel coupon.

Trimethoxysilylpropyldiethylenetriamine (2.0 g), dimethyldimethoxysilane (0.5 g), methanol (7.125 g) and deionized water (0.375 g) were added to a small vial. The solution was mixed for several minutes. A 1×6 x 0.05 cm stainless steel coupon was cleaned with methanol, then water, and finally methanol again and was attached to a Dremel® and subjected to the following procedure: dipped for 3 seconds in the above described silane solution, rotated in air for 15 seconds, dipped again for 3 seconds, rotated in air for 15 seconds, dipped once again for 3 seconds, and rotated in air for 15 seconds. The coupon was then placed in a vacuum oven at 90° C. to cure for 15 minutes under a 100 mm of Hg vacuum. After the coupon was removed from the oven and allowed to cool to room temperature, the procedure was repeated two additional times for a total of three coating cycles. Upon cooling to room temperature, the reiteratively coated coupon was placed in a test tube and immersed in acetonitrile. The tube was transferred to a Parr® hydrogenation pressure vessel and oxygen was removed from the vessel using repeated cycles of pressurization/depressurization with argon gas. This was followed by the introduction of NO at a pressure of 276 kPa (40 psi). The tube containing the coated coupon was exposed to the NO gas for 24 hours. Thereafter, the acetonitrile was decanted and the coupon repeatedly washed with a total volume of 20 mL of diethyl ether, and flushed dry under a stream of nitrogen gas. The NO content of a 1×1×0.05 cm square of the abovementioned diazeniumdiolated coated coupon was determined by immersing it in a 0.1 M phosphate buffer, pH 7.4 at 37° C., whereupon chemiluminescence-detectable NO was evolved over a 7 day period of analysis. The total NO release was measured at 13,060 pmoles/mm².

### EXAMPLE 3

This Example illustrates the preparation of a nitric oxide-releasing substituted ammonium salt of a mixed diethylenetriaminopropylpolysilane and dimethylpolysilane-coated stainless steel stent.

Trimethoxysilylpropyldiethylenetriamine (2.0 g), dimethyldimethoxysilane (0.5 g), methanol (7.125 g) and deionized water (0.375 g) were added to a small vial. The solution was mixed for several minutes. A methanol/water/methanol cleaned stainless steel S670® stent was attached to a Microman® M50 piston and subjected to the following procedure: dipped for 5 seconds in the above described silane solution, flushed under a stream of nitrogen gas at 138 kPa (20 psi) for 15 seconds, dipped again for 5 seconds, flushed under a stream of nitrogen gas at 138 kPa (20 psi) for 15 seconds, dipped once again for 5 seconds, and flushed under a stream of nitrogen gas at 138 kPa (20 psi) for 15 seconds. The stent was then placed in a vacuum oven at 100° C. to cure for 10 minutes under a 100 mm of Hg vacuum. After the coupon was removed from the oven and allowed to cool to room temperature under a blanket of nitrogen gas, the abovementioned procedure was repeated eight additional times for a total of nine coating cycles. Upon cooling to room temperature, the reiteratively coated stent was placed in a test tube and immersed in acetonitrile. The tube was then transferred to a Parr® hydrogenation pressure vessel and oxygen was removed from the vessel using repeated cycles of pressurization/depressurization with argon gas. This was followed by the introduction of NO at a pressure of 276 kPa (40 psi). The tube containing the coated stent was exposed to the NO gas for 24 hours. Thereafter, the acetonitrile was decanted and the stent was repeatedly washed with a total volume of 20 mL of diethyl ether, and flushed dry under a stream of nitrogen gas.

The NO content of the diazeniumdiolated coated stent was determined by immersing it in a 0.1 M phosphate buffer, pH 7.4 at 37° C., whereupon chemiluminescence-detectable NO was measured over several days of analysis. The total NO release was measured at 37,800 pmoles/mm².

### EXAMPLE 4

This Example illustrates the preparation of a nitric oxide-releasing substituted ammonium salt of diethylenetriaminopropylpolysilane-coated borosilicate glass.

Trimethoxysilylpropyldiethylenetriamine (2.5 g), methanol (7.125 g) and deionized water (0.375 g) are added to a small vial. The solution is mixed for several minutes. A methanol/water/methanol cleaned 1×6 cm borosilicate glass coupon is attached to a Dremel® and subjected to the following procedure: dipped for 3 seconds in the above-described silane solution, rotated in air for 15 seconds, dipped again for 3 seconds, rotated in air for 15 seconds, dipped once again for 3 seconds, and rotated in air for 15 seconds. The coupon is then placed in a vacuum oven at 90° C. to cure for 15 minutes under a 100 mm of Hg vacuum. After the coupon is removed from the oven and allowed to cool to room temperature, the procedure is repeated two additional times. Upon cooling to room temperature, the reiteratively coated coupon is placed in a test tube and immersed in acetonitrile. The tube is then transferred to a Parr® hydrogenation pressure vessel and oxygen is removed from the vessel using repeated cycles of pressurization/depressurization with argon gas. This is followed by the introduction of NO at a pressure of 276 kPa (40 psi). The tube containing the coated coupon is exposed to the NO gas for 24 hours. Thereafter, the acetonitrile is decanted and the coupon repeatedly washed with a total volume of 20 mL of diethyl ether, and flushed dry under a stream of nitrogen gas.

The NO content of a 1×1×0.1 cm square of the above-mentioned diazeniumdiolated coated glass coupon is determined by immersing it in a 0.1 M phosphate buffer, pH 7.4 at 37° C., whereupon chemiluminescence-detectable NO is evolved over a 4 day period of analysis. The total NO release is estimated at 5,465 pmoles/mm².

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for preparing a nitric oxide-releasing substrate comprising: (a) contacting an amine-functionalized silane with the substrate to form a single layer substrate; (b) contacting the single layer substrate with at least one additional amine-functionalized silane that is the same or different to form a multi-layer substrate; and (c) contacting the multi-layer substrate with nitric oxide gas.

2. The method according to claim 1, wherein the amine-functionalized silanes are hydrolyzed in an aqueous reagent.

3. The method according to claim 1, wherein the substrate comprises metal, glass, plastic, rubber, a natural fibrous material, synthetic fibrous material, or combinations thereof.

4. The method according to claim 2, wherein the substrate comprises metal.

5. The method according to claim 4, wherein the metal is selected from the group consisting of stainless steel, gold or gold alloys, metal substrates having a gold-containing coating, titanium and titanium alloys, metal substrates having an iron or iron-containing coating, metal substrates having a titanium-containing coating, nickel or nickel alloys, metal substrates having a nickel-containing coating, silicon and silicon alloys; metal substrates having a silicon-containing coating, aluminum and aluminum alloys, metal substrates having an aluminum-containing coating, zinc and zinc alloys, metal substrates having a zinc-containing coating, magnesium alloys, tin and tin alloys, metal substrates having a tin-containing coating, copper and copper alloys, metal substrates having a copper-containing coating, and combinations thereof.

6. The method according to claim 5, wherein the metal is stainless steel.

7. The method according to claim 3, wherein the substrate comprises glass.

8. The method according to claim 7, wherein the glass is selected from the group consisting of soda lime glass, strontium glass, barium glass, borosilicate glass, glass-ceramics comprising lanthanum, and combinations thereof.

9. The method according to claim 3, wherein the substrate comprises plastic.

10. The method according to claim 9, wherein the plastic is selected from the group consisting of acrylics, acrylonitrile-butadiene-styrene, acetals, polyphenylene oxides, polyimides, polystyrene, polypropylene, polyethylene, polytetrafluoroethylene, polyvinylidene, polyethylenimine, polyesters, polyethers, polylactones, polyurethanes, polycarbonates, polyethylene terephthalate, and combinations thereof.

11. The method according to claim 3, wherein the substrate comprises rubber.

12. The method according to claim 11, wherein the rubber is selected from the group consisting of silicones, fluorosilicones, nitrile rubbers, silicone rubbers, fluorosilicone rubbers, polyisoprenes, sulfur-cured rubbers, isoprene-acrylonitrile rubbers, and combinations thereof.

13. The method according to claim 3, wherein the substrate comprises ceramic.

14. The method according to claim 13, wherein the ceramic is selected from the group consisting of alumina, silicon nitride, boron carbide, boron nitride, silica, and combinations thereof.

15. The method according to claim 3, wherein the substrate comprises a natural fibrous material.

16. The method according to claim 15, wherein the natural fibrous material is selected from the group consisting of cotton, linen, silk, hemp, wool, and combinations thereof.

17. The method according to claim 3, wherein the substrate comprises a synthetic fibrous material.

18. The method according to claim 1, wherein the amine-functionalized silane is selected from the group consisting of 3-aminopropyltrimethoxysilane, 3-aminopropyltriethoxysilane, 3-aminopropyldimethoxysilane, N-(3-acryloxy-2-hydroxypropyl)-3-amino-propyltriethoxysilane, N-2-(aminoethyl)-3-aminopropyltris(2-ethyl-hexoxy)silane, 3-(m-aminophenoxy)propyltrimethoxysilane, 3-(1-aminopropoxy)-3,3-dimethyl-1-propenyl-trimethyoxysilane, 3-aminopropyltris(methoxyethoxyethoxy)silane, 3-aminopropylmethyldiethoxysilane, 3-aminopropyltris(trimethylsiloxy)silane, bis(dimethylamino)methylchlorosilane, bis(dimethylamino)methylmethoxysilane, bis(dimethylamino)phenylchlorosilane, bis(dimethylamino)phenylethoxysilane, bis(2-hydroxyethyl)-3-aminopropyltriethoxysilane, bis(2-hydroxyethyl)-3-aminopropyltrimethoxysilane, bis(3-triethoxysilyl)propylamine, 1,4-bis[3-(trimethoxysilyl)propyl]ethylenediamine, (N,N-diethyl-3-aminopropyl)trimethoxysilane, (N,N-dimethyl-3-aminopropyl)trimethoxysilane, N-phenylaminopropyltrimethoxysilane, trimethoxysilylpropyldiethylenetrianine, trimethoxysilylpropylpentaethylenehexamine, triethoxysilyloctyldiethylenetriamine, triisopropoxysilylpentaethylenehexamine, n-trimethoxysilylpropyl-N,N,N-trimethylanmonium chloride, 3-aminopropylmethyldiethoxysilane, 2-(perfluorooctyl)ethyltriaminotrimethoxysilane, 4-aminobutyltrimethoxysilane, N-(6-aminohexyl)aminopropyltrimethoxysilane, 3-(dimethoxymethylsilylpropyl)diethylenetriamine, N-(2-aminoethyl)-N′-[3-(dimethoxymethylsilyl)propyl]-1,2-ethanediamine, amine-functionalized polydimethylsiloxane copolymer, and bis-aminosilane.

19. The method according to claim 18, wherein the bis-aminosilane is selected from the group consisting of bis-(trimethoxysilylpropyl)amine, bis-(triethoxysilylpropyl)amine, bis-(triethoxysilylpropyl)ethylene diamine, N-[2-vinylbenzylamino)ethyl]-3-aminopropyltrimethoxysilane, aminoethylaminopropyltrimethoxysilane, trimethoxysilyl-modified polyethylenimine, methyldimethoxysilyl-modified polyethylenimine, and combinations thereof.

20. The method according to claim 1, wherein the amine-functionalized silane is combined or mixed with at least one functionalized and nonfunctionalized silane selected from the group consisting of 2-acetoxyethyltrichlorosilane, 2-acetoxyethyldimethylchlorosilane, acryloxypropylmethyldimethoxysilane, 3-acryloxypropyltrichlorosilane, 3-acryloxypropyltrimethoxysilane, adamantylethyltrichlorosilane, allyldimethylchlorosilane, allyltrichlorosilane, allyltriethoxysilane, allytrimethoxysilane, amyltrichlorosilane, amyltriethoxysilane, amyltrimethoxysilane, 5-(bicycloheptenyl)methyldichlorosilane, 5-(bicycloheptenyl)methyltriethoxysilane, 5-(bicycloheptenyl)methyltrimethoxysilane, 5-(bicycloheptenyl)dimethylmethoxysilane, 5-(bicycloheptenyl)methyldiethoxysilane, bis(3-cyanopropyl)dichlorosilane, bis(3-cyanopropyl)diethoxysilane, bis(3-cyanopropyl)dimethoxysilane, 1,6-bis(trimethoxysilyl)hexane, bis(trimethylsiloxy)methylsilane, bromomethyldimethylchlorosilane, bromomethyldimethylmethoxysilane, 3-bromopropyltrichlorosilane, 3-bromopropyltriethoxysilane, n-butyldimethylchlorosilane, n-butyldimethylmethoxysilane, tert-butyldimethylchlorosilane, ter-butyldimethylisoproplysilane, tert-butyldiphenylchlorosilane, tert-diphenylmethoxysilane, n-butylmethyldichlorosilane, n-butyldimethoxysilane, n-butyldiethoxysilane, n-butyldiisopropylsilane, n-butyltrimethoxysilane, (10-carbomethoxydecyl)dimethylchlorosilane, 2-(carbomethoxy)ethyltrimethoxysilane, 4-chlorobutyldimethylmethoxysilane, 4-chlorobutyldimethylethoxysilane, 2-chloroethylmethyldiisopropylsilane, 2-chloroethyltriethoxysilane, chloromethyldimethylethoxysilane, p-(chloromethyl)phenyltriethoxysilane, p-(chloromethyl)phenyltrimethoxysilane, chloromethyltriethoxysilane, chlorophenyltrimethoxysilane, 3-chloropropylmethyldimethoxysilane, 3-chloropropyltriethoxysilane, 2-(4-chlorosulfonylphenyl)ethyltrichlorosilane, 2-cyanoethylmethyltrimethoxysilane, (cyanomethylphenethyl)triethoxysilane, 3-cyanopropyldimethyldiisopropylsilane, 2-(3-cyclohexenyl)ethyl]trimethoxysilane, cyclohexydiethoxymethylsilane, cyclopentyltrimethoxysilane, di-t-butoxydiacetoxysilane, di-n-butyldimethoxysilane, dicyclopentyldimethoxysilane, diethyldiethoxysilane, diethyldimethoxysilane, diethyldibutoxysilane, diethylphophatoethyltriethoxysilane, diethyl(triethoxysilylpropyl)malonate, di-n-hexyldimethoxysilane, diisopropyldichlorosilane, diisopropyldimethoxysilane, dimethyldiacetoxysilane, dimethyldimethoxysilane, 2,3-dimethylpropyldimethylethoxysilane, dimethylethoxysilane, dimethylmethoxychlorosilane, dimethyl-n-octadecylchlorsilane, N,N-dimethyltriethylsilylamine, 1,3-diemethyltetramethoxydisoloxane, diphenylchlorosilane, diphenyldiacetoxysilane, diphenydiethoxysilane, diphenyldifluorosilane, diphenyldimethoxysilane, diphenylmethylchlorosilane, diphenylmethylethoxysilane, 2-(diphenylphosphino)ethyltriethoxysilane, divinylethoxysilane, divinyldichlorosilane, n-docosylmethyldichlorosilane, n-dodecyltriethoxysilane, 2-(3,4-epoxycyclohexyl)ethyltrimethoxysilane, ethyldimethylchlorosilane, ethyltriacetoxysilane, ethyltriethoxysilane, ethyltrimethoxysilane, 3-glycidoxypropyldimethylethoxysilane, (3-glycidoxypropyl)methyldimethoxysilane, 3-glycidoxypropyltrimethoxysilane, (3-heptafluoroisopropoxy)propylmethyl-dichlorosilane, n-heptylmethyldichlorosilane, n-heptylmethyldimethoxysilane, n-hexadecyltrichlorosilane, n-hexadecyltriethoxysilane, 6-hex-1-enyltrichlorosilane, 5-hexenyltrimethoxysilane, n-hexylmethyldichlorosilane, n-hexyltrichlorosilane, n-hexytriethoxysilane, n-hexyltrimethoxysilane, 3-iodopropyltriethoxysilane, 3-iodopropyltrimethoxysilane, isobutyldimethylchlorosilane, isobutylmethyldichlorosilane, isobutyltrimethoxysilane, isobutyltriethoxysilane, 3-isocyanatopropyldimethylchlorosilane, isocyanatopropyldimethylmethoxysilane, 3-isocyanatopropyltriethoxysilane, isooctyltrichlorsilane, isooctyltriethoxysilane, isopropyldimethylchlorosilane, 3-mercaptopropylmethyldimethoxysilane, 3-mercaptopropyltrimethoxysilane, 3-mercaptopropyltriethoxysilane, 3-methacryloxypropylmethyldiethoxysilane, 3-methacryloxypropylmethyldimethoxysilane, 3-methacryloxypropyltrimethoxysilane, 3-(4-methoxyphenyl)propyltrichlorosilane, 3-(4-methoxyphenyl)propyltrimethoxysilane, methylcyclohexyldichlorosilane, methylcyclohexyldiethoxysilane, methyldiacetoxysilane, methyldichlorosilane, methyldiethoxysilane, methyldimethoxysilane, methyldodecyldichlorosilane, methyldodecyldiethoxysilane, methylisopropyldichlorosilane, methyl-n-octadecyldimethoxysilane, methyl-n-octyldichlorosilane, (p-methylphenethyl)methyldichlorosilane, methyl(2-phenethyl)dimethoxysilane, methylphenyldiisopropoxysilane, methylphenyldiethoxysilane, methylphenyldimethoxysilane, methyl-n-propyldimethoxysilane, methyltriacetoxysilane, methyltriethoxysilane, neophylmethyldiethoxysilane, n-octadecyldimethylmethoxysilane, n-octadecyltriethoxysilane, n-octadecyltrimethoxysilane, 7-oct-1-enylmethylchlorosilane, 7-oct-enyltrimethoxysilane, n-octyldiisopropylchlorosilane, n-octyldimethylchlorosilane, n-octylmethyldimethoxysilane, n-octyltriethoxysilane, 1,1,1,3,3-pentamethyl-3-acetoxydisiloxane, phenethyldimethylchlorosilane, phenethyldimethylmethoxysilane, phenethyltriethoxysilane, phenyl(3-chloropropyl)dichlorosilane, phenyldimethylacetoxysilane, phenyldimethylethoxysilane, phenylmethylvinylchlorosilane, (3-phenylpropyl)dimethylchlorosilane, phenyltriethoxysilane, phenyltrimethoxysilane, phthalocyanatodimethoxysilane, n-propyldimethylchlorosilane, n-propyltrimethoxysilane, styrylethyltrimethoxysilane, tetra-n-butoxysilane, tetraethoxysilane, tetramethoxysilane, tetraproproxysilane, (tridecafluoro-1,1,2,2,-tretrahydrooctyl)-1-trimethoxysilane, triethoxysilane, triethoxysilylpropylethyl carbamate, triethylacetoxysilane, triethylethoxysilane, (3,3,3-trifluoropropyl)dimethylchlorosilane, (3,3,3-trifluoropropyl)methyldimethoxysilane, (3,3,3-trifluoropropyl)triethoxysilane, triisopropylchlorosilane, trimethoxysilane, 1-trimethoxysilyl-2-(p,m-chloromethyl)-phenylethane, trimethylethoxysilane, 2-(trimethylsiloxy)ethyl methacrylate, p-trimethylsiloxynitrobenzene, o-trimethylsilylacetate, triphenylethoxysilane, n-undeceyltrimethoxysilane, vinyldimethylethoxysilane, vinyltriacetoxysilane, vinyltrimethoxysilane, and combinations thereof.

21. The method according to claim 1, wherein the nitric oxide-releasing substrate comprises a nitric oxide-releasing functional group that is an O²-protected diazeniumdiolate of an amine-functionalized silane.

22. The method according to claim 1, wherein the amine-functionalized silane is dissolved in a solvent or solvent mixture containing at least one molar equivalent of water.

23. The method according to claim 1, wherein the amine-moiety of the amine-functionalized silane is selected from the group consisting of diethylenetriamine, pentaethylenehexamine, low and high molecular weight linear/branched polyethylenimines, amine-functionalized divinylbenzene, piperazine, and combinations thereof.

24. The method according to claim 1, further comprising: prior to (d), treating the amine-functionalized siliceous substrate with a biocompatible topcoat.

25. The method according to claim 24, wherein the biocompatible topcoat is a lubricious hydrogel.

26. The method according to claim 25, wherein the lubricious hydrogel is selected from the group consisting of homo- and heteropolyethers, polyols, polyureas, polylactones, albumin-, heparin-, and polyphosphorylcholine-functionalized polymers, and combinations thereof.

27. A method for preparing a nitric oxide-releasing substrate comprising: (a) contacting a substrate with an amine-functionalized silane; (b) contacting the substrate with at least one additional amine-functionalized silane; and (c) contacting the substrate with a nitric oxide-releasing functional group to form an NO-releasing substrate.

28. A method for preparing a nitric oxide-releasing substrate comprising: (a) contacting a substrate with an amine-functionalized silane; (b) contacting the substrate with at least one additional amine-functionalized silane that is the same or different that is the same or different; (c) contacting the substrate with a nucleophile, and (d) contacting the substrate with nitric oxide to form an NO-releasing substrate.

29. A nitric oxide-releasing substrate prepared according to the method of claim 1.

30. A nitric oxide-releasing substrate prepared according to the method of claim 26.

31. A nitric oxide-releasing substrate prepared according to the method of claim 27.

32. A nitric oxide-releasing substrate having nitric oxide bonded thereto through a NO-releasing nucleophile residue bonded to a polysilane coating, wherein the polysilane coating is bonded to the substrate and comprises at least one amine-functionalized silane.

33. The substrate according to any of claims 29, 30, 31, or 32, wherein the substrate is part of a medical device.

34. The substrate according to claim 33, wherein the medical device comprises a metal.

35. The substrate according to claim 34, wherein the metal of the medical device comprises stainless steel.

36. The substrate according to claim 33, wherein the medical device is selected from the group consisting of an arterial stent, guide wire, catheter, trocar needle, bone anchor, bone screw, protective plating, hip and joint implant, electrical lead, sensor, probe, blood oxygenator, blood pump, blood storage bag, blood collection tube, blood filter including filtration media, tubing, pacemaker, pacemaker leads, heart valves, pulse generator, cardiac defibrillator, cardioverter defibrillator, spinal stimulator, brain and nerve stimulator, introducer, amniocentesis and biopsy needles, cannulae, drainage tubes, shunts, transducers, implants, specula, irrigators, nozzles, calipers, forceps, retractors, vascular grafts, personal hygiene items, absorbable and nonabsorbable sutures, and wound dressings.

37. A nitric oxide-releasing substrate comprising a polysilane coating comprising at least two layers of amine-functionalized silane and a nitric oxide-releasing N₂O₂⁻ group.

38. The method of claim 1, wherein said amine-functionalized silane and said at least one additional amine-functionalized silane are the same.

39. The method of claim 1, wherein said amine-functionalized silane and said at least one additional amine-functionalized silane are different.

40. A method for preparing a nitric oxide-releasing material comprising contacting a first amine-functionalized silane with at least one additional amine-functionalized silane and contacting the amine-functionalized silane with nitric oxide gas.

41. The method of claim 40, wherein the first amine-functionalized silane and said at least one additional amine-functionalized silane are the same.

42. The method of claim 40, wherein the first amine-functionalized silane and said at least one additional amine-functionalized silane are different.

43. The method of any of claims 40-42, wherein an additive is contacted with the first amine-functionalized silane.

44. A nitric oxide-releasing material prepared according to the method of any of claims 40-43.
